(19) United States
(12) Patent Application Publication
(10) Pub. No.: US 2022/0016372 A1
(43) Pub. Date: Jan. 20, 2022

(54) SUCTION-ENABLED STYLET FOR ENDOTRACHEAL TUBE

(71) Applicant: Brantleymarie Medical Devices, Inc., Mt. Juliet, TN (US)

(72) Inventors: Elizabeth Baldwin, Charlotte, TN (US); Daniel Hines, Mt. Juliet, TN (US)

(73) Assignee: Brantleymarie Medical Devices, Inc., Mt. Juliet, TN (US)

(21) Appl. No.: 17/113,806

(22) Filed: Dec. 7, 2020

Related U.S. Application Data
(60) Provisional application No. 63/051,540, filed on Jul. 14, 2020.

(51) Int. Cl. A61M 16/04; A61M 16/20

(52) U.S. Cl. CPC A61M 16/0463; A61M 16/208; A61M 16/0402

(57) ABSTRACT

A stylet for use with a suction system and with an endotracheal (ET) tube configured for insertion into a patient airway in an intubation procedure. The stylet has a semi-rigid elongate tube portion and is configured for removable insertion into the ET tube. An inlet is located at a first end of the stylet and an outlet is located at a second end of the stylet. A suction interface located at the second end of the stylet interfaces with the suction system. The stylet is configured such that a suction pressure created within the suction passage by the suction system suctions substances located within the patient airway into the stylet via the inlet, through the suction passage, and then out of the stylet via the outlet.

### CROSS REFERENCE TO RELATED APPLICATIONS

This application claims the benefit of U.S. Provisional Patent Application No. 63/051,540, filed on Jul. 14, 2020 and entitled ENDOTRACHEAL TUBE: SUCTION-ENABLED STYLET, which is incorporated herein by reference in its entirety.

### FIELD

The present invention relates generally to medical devices. More particularly, the present invention relates to a suction-enabled stylet for use in conjunction with an endotracheal tube for intubation.

### BACKGROUND

Some medical procedures are invasive and potentially dangerous although they are necessary life-saving procedures. Intubation, specifically endotracheal intubation, is typically performed for various medical conditions or procedures, such as the application of general anesthesia. With initial reference to FIGS. 1-3, endotracheal intubation involves the placement or the insertion of an endotracheal (ET) tube 100 into a patient's trachea T to protect the patient's airway and provide a means of mechanical ventilation. The ET tube 100 has a patient end 118 that is sized to pass into a patient airway and then into the trachea T. Additionally, the ET tube 100 has a user end 120, located opposite the patient end 118, which is typically grasped by the user while performing the intubation. Delay or misplacement of the ET tube 100, such as misplacement of the ET tube into esophagus E, may cause permanent neurological damage or death. Incorrect positioning of the ET tube 100 may also jeopardize airway protection or cause inadequate ventilation. It is, therefore, imperative to intubate a patient quickly and position the ET tube 100 correctly when a medical condition arises.

Various technologies have been developed to assist with the placement of the ET tube 100 into the trachea T in an intubation procedure. For example, when performing a technique called “direct laryngoscopy,” a practitioner holds a laryngoscope 102 in his left hand and uses the laryngoscope to push down the tongue and lift up the epiglottis in order to obtain a direct view of the glottis G and airway. At the same time, the practitioner holds the ET tube 100 in his right hand and then passes it through the mouth, larynx, and vocal cords into the trachea T under direct vision. However, prior to inserting the ET tube 100 into the patient's airway, a thin, solid, and rigid or semi-rigid stylet 104 is inserted into the ET tube. The stylet 104 is used to help provide rigidity and guidance to the ET tube 100 as it is guided to the trachea T. The stylet 104 is slightly bent and that bend guides the end of the ET tube 100 upwards (as seen in FIG. 3) through the curved airway, through the vocal cords, into the trachea T and away from esophagus E. The practitioner typically withdraws the laryngoscope 102 to free his left hand, and advances the endotracheal tube down the trachea T into place. Lastly, the stylet 104 is withdrawn from the ET tube 100, which remains in place in the patient's trachea. The practitioner then inflates a small cuff 106 near the end of the ET tube 100, inside the trachea T, to prevent backflow and leakage of air, and starts pumping air into and out of the ET tube.

Maintaining a clear “picture” or view of the airway is critical to a safe, successful and fast intubation procedure. As discussed above, the laryngoscope 102 and stylet 104 are important tools for maintaining a clear view of the airway. However, despite the improved visibility provided by these tools, failed intubations (e.g., incorrect placement into the esophagus) and slow intubations occur frequently. One reason for these failed and slow intubations is that visual verification may be made difficult or impossible due to heavy airway secretions or bleeding within the patient's airway. With reference to FIG. 4, this issue is frequently addressed through the use of a suction system, such as wall-mounted suction systems 108 that are frequently found in hospitals. These suction systems 108 typically include a regulator 110 for controlling the suction pressure, a canister 112 for collecting collected substances, and a hose 114 that may include a wand 116 that is held by the practitioner in carrying out the suction procedure. As discussed above, in a typical intubation procedure, the practitioner holds the laryngoscope in one hand and the ET tube in other hand. Thus, when suction is required while performing an intubation, the practitioner would have to free one hand, such as by withdrawing or releasing his grip on the ET tube, in order to hold and use the suction device. Alternatively, if another person, such as an assistant, is available, that assistant could carry out the suction process while the practitioner continues to hold the laryngoscope and ET tube. Either option adds time and complexity to the intubation procedure. Additionally, either option requires the ET tube to be withdrawn and reinserted into the patient airway or for the hose 114 or wand 116 to be inserted and then withdrawn from the airway. This increases the chances for further injuring the patient, such as the patient's vocal cords (extremely delicate tissue).

What is needed, therefore, is a method and apparatus for carrying out an intubation procedure that enables a clear view of the airway to be maintained and that addresses the issues discussed above.

### Notes on Construction

The use of the terms “a”, “an”, “the” and similar terms in the context of describing embodiments of the invention are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. The terms “substantially”, “generally” and other words of degree are relative modifiers intended to indicate permissible variation from the characteristic so modified. The use of such terms in describing a physical or functional characteristic of the invention is not intended to limit such characteristic to the absolute value which the term modifies, but rather to provide an approximation of the value of such physical or functional characteristic.

Terms concerning attachments, coupling and the like, such as “attached”, “connected” and “interconnected”, refer to a relationship wherein structures are secured or attached to one another either directly or indirectly through intervening structures, as well as both moveable and rigid attachments or relationships, unless otherwise specified herein or clearly indicated as having a different relationship by context. The term “operatively connected” is such an attachment, coupling or connection that allows the pertinent structures to operate as intended by virtue of that relationship.

The use of any and all examples or exemplary language (e.g., “such as” and “preferably”) herein is intended merely to better illuminate the invention and the preferred embodiments thereof, and not to place a limitation on the scope of the invention. Nothing in the specification should be construed as indicating any element as essential to the practice of the invention unless so stated with specificity.

As used herein, the term “semi rigid” means deformable but shape retaining. As used herein, the term “semi rigid” also means stiff and solid but not inflexible.

As used herein, the term “airflow” means a flow of gas, which can include atmospheric air or other gases or combinations of gases (e.g., medical-grade oxygen, anesthetic gases, etc.).

As used herein, the term “airway” generally means the upper airway and includes the trachea, larynx, and pharynx.

As used herein, the term “maximum suction pressure” means the maximum suction pressure that is available for a given setting of the suction system.

### SUMMARY

The above and other needs are met by a stylet that is configured for use with a suction system and with an endotracheal (ET) tube having a patient end that is sized to pass into a patient airway and a user end that is located opposite the patient end. Also disclosed herein is an intubation system that is composed of the stylet and ET tube. The stylet includes a first end configured for removable insertion into an opening located at the user end of the ET tube and a second end that is located opposite the first end of the stylet. A semi-rigid elongate tube portion connects the first end and the second end of the stylet together. The tube portion is sized to slide into the ET tube. An inlet is located at the first end of the stylet and an outlet is located at the second end of the stylet. A suction passage is formed within the stylet and extends from the inlet to the outlet, including through the tube portion. A suction interface is located at the second end of the stylet and is configured to interface with the suction system. The stylet is configured such that a suction pressure created within the suction passage by the suction system suctions substances located within the patient airway into the stylet via inlet, through the suction passage, and then out of the stylet via the outlet.

In preferred embodiments, the stylet includes a suction state selector, wherein a first suction pressure is provided at the first end of the stylet when the suction state selector is operating in a first mode. Additionally, a second suction pressure that is different from the first suction pressure is provided at the first end of the stylet when the suction state selector is operating in a second mode. In certain embodiments, a maximum suction pressure is provided at the first end of the stylet when the suction state selector is operating in the first mode. In some cases, the suction pressure at the first end of the stylet is equal to or approximately equal to zero when the suction state selector is operating in the second mode. In other cases, an intermediate suction pressure that is between the minimum and maximum suction pressure is provided at the first end of the stylet when the suction state selector is operating in a third mode of operation.

The suction state selector may include a thumb valve located on the suction interface. The thumb valve provides a valve opening in the suction passage at a location between the inlet and the outlet of the stylet. The thumb valve operates in the first mode when the valve opening is sealed to prevent airflow through the valve opening and the second mode when the valve opening is fully uncovered. Additionally, the thumb valve operates in the third mode when the valve opening of the thumb valve is at least partially unsealed such that airflow through the valve opening is permitted. In certain embodiments, the suction system includes a hose and the suction interface includes a hose barb having an outlet and is configured for insertion into an end of the hose. Preferably, the tube extends away from the valve opening at an angle with respect to the hose barb. In some cases, an acute angle is formed between a midline extending through the hose barb and a midline extending through the tube of the thumb valve. In certain embodiments, flanges extend laterally outwards from opposing sides of the tube and are sized and positioned such that, when a user operates the thumb valve, a separate finger of the user may be placed under each of the flanges alongside the tube while a thumb of the user may be placed over an end of the tube. The flanges may form opposing sides of an elongate flange plate that is located around the end of the tube. In some cases, the flange plate may include opposed truncated flat side edges that extend along the length of the flange plate between and connect distal edges of the flanges.

In certain preferred embodiments of the invention, the inlet includes an opening formed at a tip of the first end of the stylet. In some cases, the inlet includes a plurality of spaced apart openings disposed in a sidewall of the stylet and located near the first end of the stylet. In some cases, the stylet is sized such that the first end of the stylet passes out of an opening located at the patient end of the ET tube when the stylet is fully inserted into the ET tube. In preferred embodiments, the second end of the stylet is larger than the opening at the user end of the ET tube such that the stylet cannot slide completely into the ET tube.

Also disclosed herein is a method of intubation. The method includes the step of providing an endotracheal (ET) tube having a patient end and a user end opposite the patient end, a suction system, and a stylet. The stylet includes a first end, a second end that is located opposite the first end of the stylet, a semi-rigid elongate tube portion connecting the first end and the second end together, an inlet located at the first end of the stylet, an outlet located at the second end of the stylet, a suction passage formed within the stylet that extends from the inlet to the outlet and through the tube portion, and a suction interface disposed at the second end of the stylet. The method also includes the steps of connecting the stylet to the suction system with the suction interface and then inserting the first end of the stylet into an opening located at the user end of the ET tube. Next, while the stylet is inserted into the ET tube, the patient end of the ET tube and the stylet are guided into an airway of a patient. Next, while the stylet is inserted into the ET tube and the ET tube is located in the airway of the patient, a suction pressure is created within the suction passage using the suction system in order to suction substances from the airway. Finally, the stylet is extracted from the ET tube and the ET tube remains in the airway. In certain preferred embodiments, the first end of the stylet passes out of an opening located at the patient end of the ET tube. Then, the suctioning step is performed while the stylet is inserted into one end of the ET tube and a portion of the stylet protrudes from an opposite end of the ET tube within the patient airway.

### BRIEF DESCRIPTION OF THE DRAWINGS

Further advantages of the invention are apparent by reference to the detailed description when considered in conjunction with the figures, which are not to scale so as to more clearly show the details, wherein like reference numerals represent like elements throughout the several views, and wherein:

FIG. 1 depicts an endotracheal (ET) tube used in performing an intubation procedure;

FIG. 2 depicts a conventional stylet that may be inserted into the ET tube of FIG. 1 when performing an intubation procedure;

FIG. 3 illustrates an intubation procedure being performed with an ET tube and a laryngoscope;

FIG. 4 illustrates a suction system that may be used in performing an intubation procedure;

FIG. 5 is a perspective view depicting a suction-enabled stylet configured for use with an ET tube in performing an intubation procedure according to an embodiment of the present invention;

FIG. 6 is a sectional view of an end of the stylet of FIG. 5 that is taken along line 6-6 and that illustrates a stylet sidewall formed using a first inner material located within an outer shell formed by a second and different material;

FIG. 7 is a cutaway view illustrating first and second ends of the stylet of FIG. 5 and including a portion of a hose of a suction system connected to the second end of the stylet;

FIG. 8 depicts a stylet extending through an ET tube and into a trachea according to an embodiment of the present invention;

FIG. 9 depicts the stylet of FIG. 8 being withdrawn from the ET tube and trachea;

FIG. 10 is a perspective view of a suction interface having a thumb valve, including a flange plate, and hose barb;

FIG. 11 depicts a suction interface according to alternative embodiment of the present invention; and

FIG. 12 depicts the suction interface in FIG. 11 in use in performing an intubation procedure.

### DETAILED DESCRIPTION

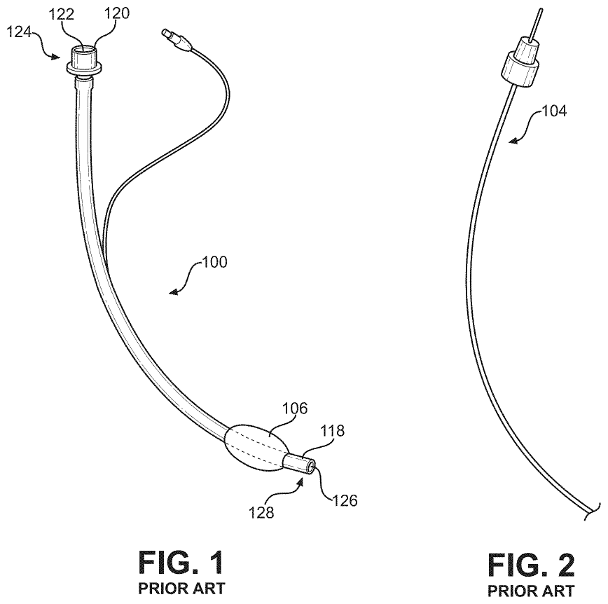

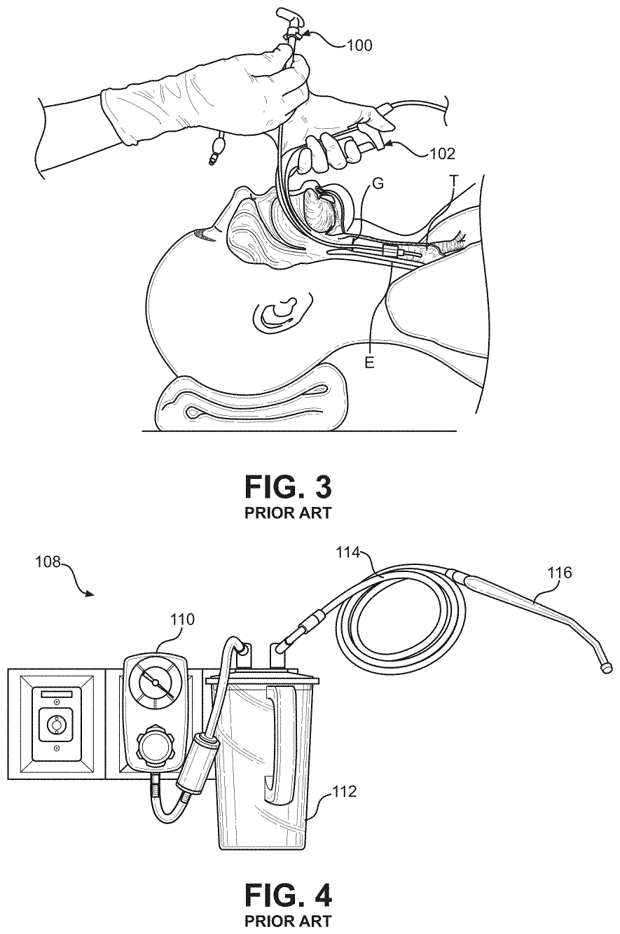

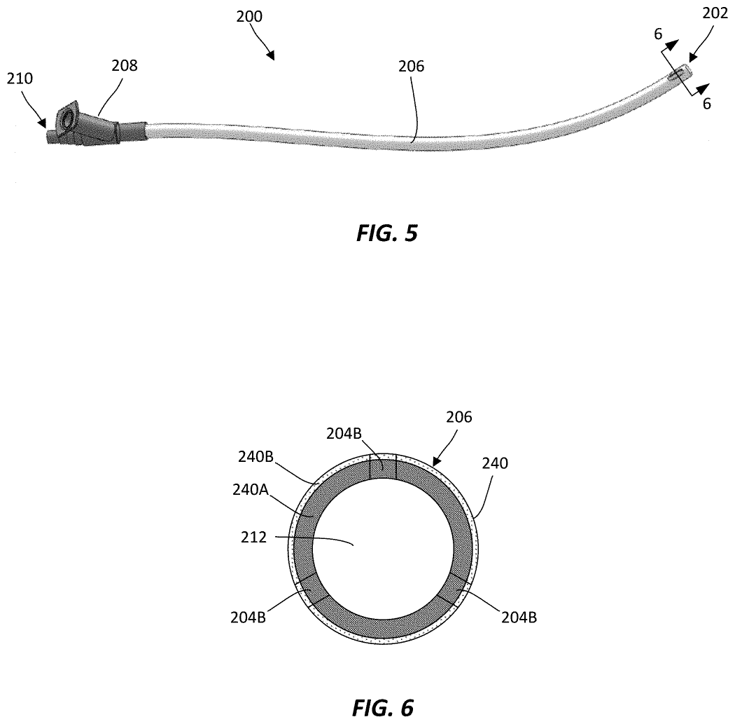

Referring now to FIG. 5, there is provided a suction enabled stylet 200 that is configured for use in connection with an endotracheal (ET) tube such as ET tube 100 (FIG. 1), with a laryngoscope such as laryngoscope 102 (FIG. 3), and with a suction system such as suction system 108 (FIG. 4) in carrying out an intubation procedure. As further detailed below, the stylet 200 is connected to the suction system 108 and then inserted into the ET tube 100. When an intubation procedure is being performed, the practitioner can hold the laryngoscope 102 in his left hand and can hold the ET tube 100 and stylet 200 in his right hand. However, unlike conventional stylets, stylet 200 serves the dual purposes of (1) providing rigidity and guiding the ET tube into the patient airway and (2) providing suction during the intubation procedure. Therefore, the practitioner can utilize the suction feature of stylet 200 immediately when needed without the need to first extract the ET tube from the patient, without the need to have another person (e.g., an assistant, nurse, etc.) provide suction, and without releasing his grip on the ET tube. Advantageously, this will enable a clear view of the patient airway to be obtained by the practitioner and for the intubation procedure to be carried out more quickly and reliably than was previously possible.

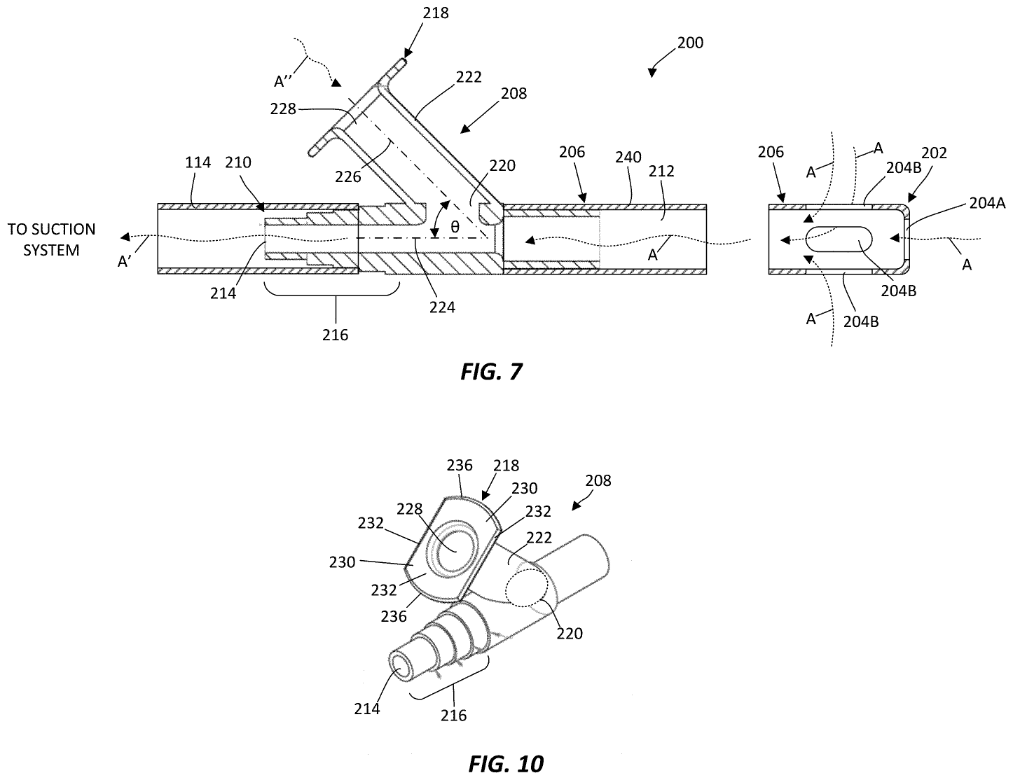

The stylet 200 has a first end 202 that is sized and configured for removable insertion into an end of the ET tube. The first end 202 of the stylet 200 includes an inlet that allows substances located within the patient airway to be suctioned into the stylet by the suction system. As shown best in FIGS. 6 and 7, in certain embodiments, the inlet is formed by an opening 204A formed at a tip of the first end 202 of the stylet 200. Additionally or alternatively, the inlet may be formed by a plurality of spaced apart openings 204B disposed in a sidewall 240 of the stylet 200 and located proximate the first end 202 of the stylet. Each of these openings 204A, 204B is sized to allow ingoing airflows A to pass into the stylet. When the stylet 200 is located within a patient airway, substances located within the airway are suctioned into the stylet via these ingoing airflows A. Preferably, the external surface of the first end 202 is rounded in order to reduce the possibility of damaging the patient's airway during the intubation procedure.

With reference to FIGS. 1, 5-7, a hollow elongate tube portion 206 extends from the first end 202 of the stylet 200 to a suction interface 208 located at a second end 210 of the stylet. Preferably, the tube portion 206 is semi rigid (but may be rigid). In certain preferred embodiments, a sidewall 240 of the tube portion 206 is formed by at least two layers of different materials that enables the tube portion to remain semi rigid. For example, sidewall 240 may include an inner layer 240A that is formed using a first material (e.g. a deformable, medical grade metal) that is located within an outer shell 240B formed by a second and different material. The shell 240B may be formed using a softer material than the material used in forming layer 240A in order to reduce the likelihood of injury to the patient. Preferably, the shell 240B is formed using a material that assists in retaining the stylet 200 and in preventing dislodgement. As such, the exterior (i.e., shell 240B) of the sidewall 240 preferably provides a greater degree of gripping or holding ability than the other portions of the sidewall, including the inner layer 240A. Of course, it should be understood that the materials selected may be varied based on manufacturer, intended application, other design considerations, etc. that are known to persons of skill in the art.

The tube portion 206 is sized to slide into an opening 122 located at a user end 124 of the ET tube 100. Preferably, the tube portion 206 of the stylet 200 is sized such that the first end of the stylet passes out of an opening 126 located at a patient end 128 of the ET tube 100 when the stylet is fully inserted into the ET tube. When the intubation procedure is performed, the patient end 128 of the ET tube 100 is passed into the patient airway first and, preferably, the user end 124 is held by the practitioner performing the procedure. Preferably, the second end 210 of the stylet 200 (or a portion thereof) is larger than the opening 122 at the user end 124 of the ET tube 100 such that the stylet 200 cannot slide completely into the ET tube. By ensuring that this larger portion is located adjacent the user end 124 of the ET tube 100, the practitioner can be assured that the first end 202 of the stylet 200 is extending out of the opposing patient end 128 of the ET tube 100.

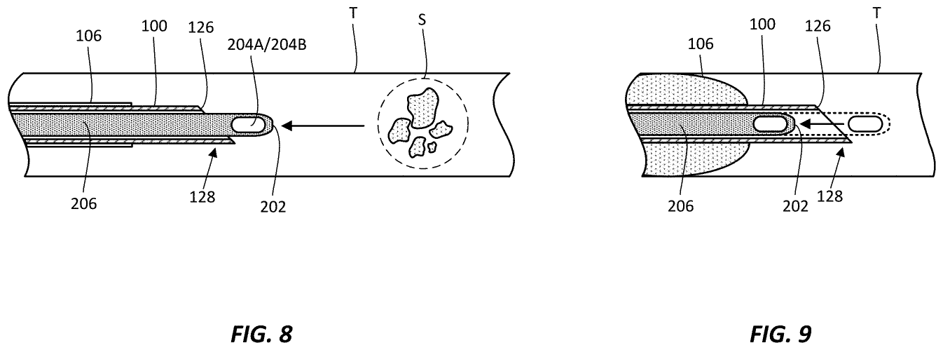

With reference to FIG. 8, once the stylet 200 is fully inserted into the ET tube 100, as discussed above, the combination is inserted into the airway (such as into trachea T). Since the first end 202 of the stylet 200 extends out through the patient end 128 of the ET tube 100, the openings 204A, 204B reach substances S (e.g., blood, secretions, etc.) located in the airway before those substances enter completely into the ET tube. This preferably allows those substances S to be suctioned directly into the stylet 200 without any of those substances being suctioned into the ET tube 100 first and then suctioned into the stylet. This assists in keeping the ET tube 100 clean and free from obstruction, which improves its effectiveness. Additionally, this allows the airway to be suctioned at the same time that the ET tube 100 is being inserted into the airway by a single person. Additionally, this suctioning action takes place in an area that is directly in front of and around the entire patient end 128 of the ET tube 100. Conventional methods merely provided suction where the end of the suction tubing was located. Therefore, in order to suction in front of the ET tube and around its periphery, substantial movement of the suction tubing was required, which increased the amount of time required for the procedure to be carried out and the potential damage to the patient's airway. As shown in FIG. 9, once the ET tube 100 has been correctly located within the airway (trachea T), the cuff 106 is inflated and the stylet 200 is extracted from the ET tube while the ET tube remains in the airway.

Referring again to FIGS. 5 and 7 and with further reference to FIG. 10, the suction interface 208 is joined with the tube portion 206 and interfaces with the suction system at the second end 210 of the stylet 200. Ingoing airflow A that enters the stylet 200 via openings 204A, 204B passes through a suction passage 212 that is formed within the tube portion 206 of the stylet and that extends from the inlet to an outlet 214 located at the second end of the stylet. In this particular case, the suction interface 208 includes a hose barb 216 that is configured for insertion into an end of a hose such as hose 114 (FIG. 4) of a suction system. The outlet 214 of the suction passage 212 is formed in the hose barb 216. As such, the stylet 200 may be directly connected to suction systems, including those wall-mounted suction systems 108 that are typically found in hospitals, as a replacement for wands 116 without requiring any other interface or equipment. Accordingly, when the suction system is activated, a suction pressure is created within the suction passage 212, which suctions substances located within the patient airway into the stylet via inlet (e.g., via openings 204A, 204B), through the suction passage, and then out of the stylet 200 (i.e., outgoing airflow A′) via the outlet 214 to the suction system (e.g., to canister 112).

In most instances, when an intubation procedure is being performed, it is desirable to have the suction system available for use at all times. However, suction is not required throughout the entire intubation procedure. In particular, suction is needed when substances are located within the airway but is not needed once those substances have been removed from the airway and a clear view of the airway is available. For that reason, intermittent use of the suction system is often necessary. Accordingly, in preferred embodiments of the present invention, the stylet 200 can be used with the suction system 108 in an active state but where a suction pressure is not present or where a reduced suction pressure (i.e., less than a maximum) is present at the first end 202 of the stylet.

Accordingly, in certain preferred embodiments, the stylet 200 includes a suction state selector for providing a first suction pressure at the first end 202 of the stylet when the suction state selector is operating in a first mode and for providing a second and different suction pressure at the first end of the stylet when the suction state selector is operating in a second mode. For example, in certain embodiments, a maximum suction pressure is provided when the suction state selector is operating in the first mode. This first mode could be used when the practitioner is actively suctioning substances from the patient airway. Of course, if the settings of the suction system are modified, such as if the settings of the regulator 110 are modified, the maximum suction pressure will also change. In certain embodiments, the suction pressure at the first end 202 of the stylet 200 is equal to zero or approximately zero when the suction state selector is operating in the second mode. This second mode could be used when suction is not required, such as when the airway is clear. In other embodiments, the suction state selector may be operated in a third mode of operation where the suction pressure is between the suction pressure in the first or second modes of operation (i.e., an intermediate suction pressure). For example, in a first mode of operation, a maximum or near maximum suction pressure (e.g., 70-100% of max) may be provided at the first end 202 of the stylet 200. In a second mode of operation, a minimum or near minimum suction pressure (e.g., 0-30% of max) may be provided at the first end 202 of the stylet 200. In a third mode of operation, an intermediate suction pressure (e.g., 30-70% of max) may be provided at the first end 202 of the stylet 200. In certain embodiments, the stylet 200 may be provided with a suction state selector having more than one intermediate mode of operation for selecting varying amounts of suction pressure. It is also noted that the operation of the stylet 200 and suction state selector would be similar if a “negative” suction pressure (i.e., blowing pressures) were utilized.

The suction state selector is preferably easily accessible to the practitioner performing the intubation procedure so that he can immediately modulate the suction pressure when needed without the assistance of another person and without releasing ET tube 100 and stylet 200 combination in his right hand laryngoscope 102 in his left hand. As shown in FIGS. 7 and 10, in the illustrated embodiment, the suction state selector preferably includes a thumb valve 218 that provides a valve opening 220 in the suction passage 212 in a location between the inlet (e.g., openings 204A, 204B) and the outlet 214 of the stylet 200. In preferred embodiments, when the suction state selector is operating in a first mode and the suction system is active, the valve opening 220 is fully covered and outgoing airflow A′ is comprised solely of ingoing airflow A through openings 204A and 204B. In that case, a maximum suction pressure is provided at the first end 202 of the stylet 200 via opening 204A, 204B.

When the suction state selector is operating in a different mode of operation and the valve opening 220 is partially uncovered, outgoing airflow A′ may be composed of ingoing airflow A through openings 204A and 204B as well as ingoing airflow A″ through the valve opening 220. In that case, a reduced suction pressure is provided at the first end 202. Similarly, if the valve opening 220 is left fully uncovered, outgoing airflow A′ is composed of ingoing airflow A through openings 204A and 204B as well as ingoing airflow A″ through the valve opening 220. In this second case, a minimal suction pressure is preferably provided at the first end 202 of the stylet 200. In certain embodiments, the suction pressure at the first end 202 is equal to zero or approximately equal to zero. It may be appreciated, therefore, that the suction pressure provided at the first end 202 of the stylet 200 may be varied by varying the amount of the valve opening 220 that is open. There are many ways of throttling or varying the opening size (i.e., percent open) known in the art, including various flow rate adjustment devices, etc. The term “thumb valve” should be interpreted to include any such suitable throttle flow rate adjustment devices.

However, in most cases, when an intubation procedure is being performed, either maximum or minimum suction is desired. Therefore, in preferred embodiments, the stylet enables the suction pressure at the first end 202 of the stylet 200 to be quickly (i.e., instantaneously or almost instantaneously) changed from a maximum suction pressure (e.g., valve opening fully covered) to a minimum suction pressure (e.g., valve opening fully opened). In this embodiment, a tube 222 extends away from valve opening 220 and a thumb opening 228 is located at an end of the tube that is opposite the valve opening 220. By placing a thumb, finger, or other covering (e.g., palm, closure, etc.) over the thumb opening 228, tube 222 and the valve opening 220 are both almost instantly sealed and a maximum pressure develops at the first end 202 of the stylet 200. On the other hand, uncovering the thumb opening 228 almost instantly minimizes the suction pressure at the first end 202 of the stylet. Thus, by covering and uncovering the thumb opening 228, the practitioner can provide intermittent maximum and minimum suction pressure to enable suction only when needed.

In certain embodiments, flanges 230 extend laterally outwards from opposing sides of the tube 222 and form opposing sides of an elongate flange plate 232 that is located around the end of the tube and thumb opening 228. The flange plate 232 includes opposed truncated flat side edges 234 that extend along the length of the flange plate between and connect distal edges 236 of the flanges. The flanges 230 and flange plate 232 provide a convenient gripping location for gripping the stylet 200 in order to remove it from the ET tube 100 and also for providing a very tight closure of the thumb opening 228. In particular, the flanges 230 are sized and positioned such that, if desired, when a user places a thumb over the thumb opening 228, a separate finger of the user may also be placed under each of the flanges alongside the tube 222, which enables the thumb valve 218 to be firmly grasped by applying pressure to opposite sides of the flange plate 230.

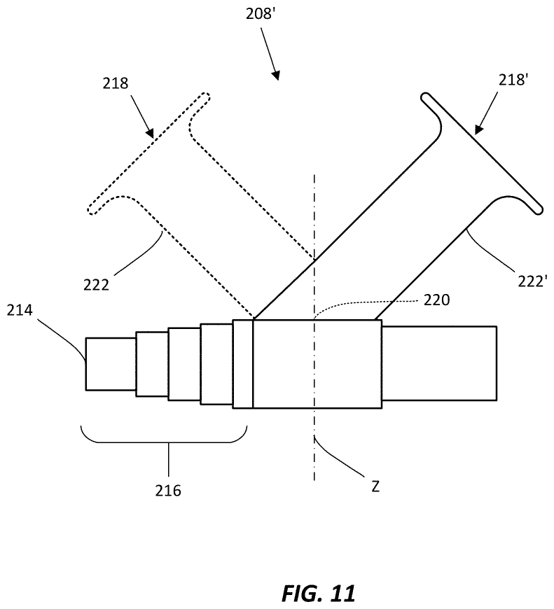

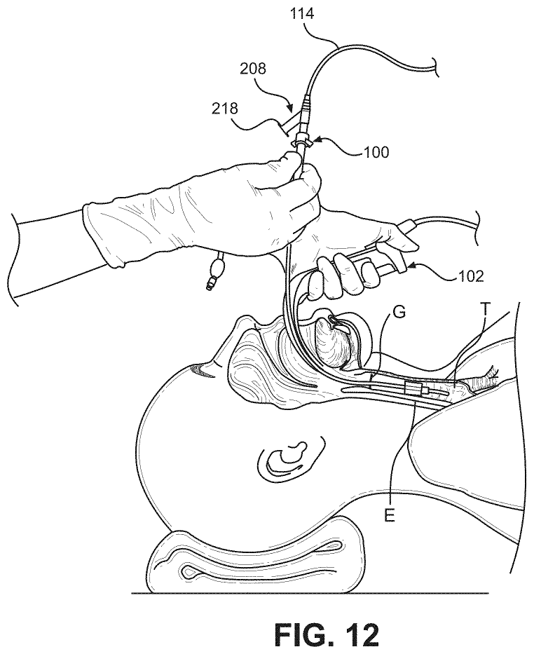

Preferably, the tube 222 is oriented such that the thumb opening 228 is easily accessible to the practitioner performing the intubation procedure. Therefore, with reference to FIG. 7, in certain embodiments, the tube 222 is angled with respect to the hose barb 216 such that the thumb opening 228 is spaced away from the outlet 214. In certain embodiments, an angle ⊖ of less than 90 degrees is formed between a midline 224 extending through the hose barb 216 and a midline 226 extending through the tube 222 of the thumb valve 218. In the embodiment illustrated in FIG. 7, the angle ⊖ between the midlines 224, 226 is approximately 45 degrees. In this way, the tube 222 extends in the same direction with the barb 216. However, in other embodiments, angle ⊖ is greater than 90 degrees such that the tube 222 extends in the opposite direction as the barb 216. With reference to FIGS. 11 and 12, in certain embodiments, the tube 222 is rotatably mounted to suction interface 208′, such that the tube rotates about an axis Z that preferably passes through the center of the valve opening 220 (general location shown by dashed lines in FIG. 11). This would enable the tube 222 to be rotated to any orientation around the valve opening 220, including to a position 222′ where it extends in a direction opposite the barb 216 and the thumb valve faces the opposite direction (i.e., to a position shown as 218′). As shown in FIG. 12, this facilitates easy access by the practitioner to the thumb valve 218′, as needed, during the intubation procedure while still maintaining control of the ET tube 100 and the laryngoscope 102. However, the preferred orientation of the thumb valve 218 is shown by dashed lines in FIG. 11, such that the opening 228 located at the end of the tube 222 may be easily placed under the practitioner's thumb when the stylet 200 is in use. For example, in the case shown in FIG. 12, the thumb valve would be directed upwards away from the patient (i.e., opposite the direction of thumb valve 218′, which is shown directed downwards towards the patient).

Although this description contains many specifics, these should not be construed as limiting the scope of the invention but as merely providing illustrations of some of the presently preferred embodiments thereof, as well as the best mode contemplated by the inventor of carrying out the invention. The invention, as described herein, is susceptible to various modifications and adaptations as would be appreciated by those having ordinary skill in the art to which the invention relates.

What is claimed is:

1. (canceled)

2. (canceled)

3. (canceled)

4. The stylet of claim 23 wherein the thumb valve functions as a suction state selector for selecting the first or second mode of operation and wherein, in the first mode of operation, a first suction pressure at the at least one opening is a maximum suction pressure and, in the second mode of operation, a second suction pressure at the at least one opening is a minimum suction pressure.

5. The stylet of claim 4 wherein the suction state selector modulates a suction pressure that is between the first and second suction pressures when the stylet is operating in a third mode.

6. The stylet of claim 5 stylet operates in the first mode when the valve opening is sealed to prevent airflow through the valve opening, the stylet operates in the second mode when the valve opening of the thumb valve is fully uncovered, and the stylet operates in the third mode when the valve opening is partially uncovered.

7. (canceled)

8. (canceled)

9. The stylet of claim 23 wherein flanges extend laterally outwards from opposing sides of the tube of the thumb valve and are sized and positioned to permit a separate finger of the user to be placed under each of the flanges alongside the tube of the thumb valve while a thumb of the user is placed over an end of the tube.

10. The stylet of claim 9 wherein the flanges form opposing sides of an elongate flange plate that is located around the end of the tube of the thumb valve, wherein the flange plate includes opposed truncated flat side edges that extend along the length of the flange plate between and connect distal edges of the flanges.

11. (canceled)

12. (canceled)

13. The stylet of claim 23 wherein the stylet is sized such that the first end of the stylet passes out of an opening located at the patient end of the ET tube when the stylet is fully inserted into the ET tube.

14. (canceled)

15. The stylet of claim 23 wherein the semi-rigid elongate tube portion is comprised of a first inner material located within an outer shell formed by a second and different material that is located on top of an outer surface of the first inner material.

16. An intubation system comprising the stylet of claim 23 and said ET tube.

17. (canceled)

18. (canceled)

19. (canceled)

20. (canceled)

21. (canceled)

22. (canceled)

23. A stylet sized and configured for use in connection with a suction system and in connection with an endotracheal (ET) tube having a patient end that is sized to pass into a patient airway and a user end that is located opposite the patient end, the stylet comprising:
a semi-rigid elongate tube portion having:
a tip located at a first end of the stylet that is configured to slide into an opening located at the user end of the ET tube;
an inlet formed by at least one opening formed in a sidewall of the tube portion and located proximate the tip;
a suction interface connected to the elongate tube portion and having:
a hose barb configured to interface with the suction system;
an outlet provided in the hose barb and located at a second end of the stylet;
a first midline beginning at an intersection point and extending along a center of the hose barb towards the outlet;
a thumb valve having:
a valve opening located longitudinally between the inlet and outlet;
a tube extending away from the valve opening;
a thumb opening located at an end of the tube that is opposite the valve opening;
a second midline beginning at the intersection point and extending through a center of the valve opening and into the tube towards the thumb opening, wherein the intersection point is located at an intersection of the first midline with the second midline, and wherein an angle Θ taken at the intersection point and formed between the first and second midlines is less than 90 degrees;
a suction passage that extends from inlet to the outlet, wherein the stylet is configured such that, in a first mode of operation, airflow does not enter the suction interface via the thumb opening and a suction pressure created within the suction interface by the suction system suctions substances located within the patient airway into the at least one opening, through the tube portion, and then into the suction interface in a first ingoing airflow traveling in a first travel direction, longitudinally through the suction interface past the valve opening in the first travel direction, and then out of the suction interface in an outgoing airflow traveling in the first direction, and
wherein the stylet is configured such that, in a second mode of operation, airflow may enter the suction interface via the thumb opening in a second ingoing airflow traveling in a second travel direction that is different from the first travel direction, and then out of the suction interface in the outgoing airflow traveling in the first travel direction.

24. The stylet of claim 23 wherein the inlet of the tube portion is comprised of two or more openings formed in a sidewall of the semi-rigid tube in a spaced apart arrangement proximate the tip of the semi-rigid tube.

25. (canceled)

26. (canceled)

27. A stylet sized and configured for use in connection with a suction system and in connection with an endotracheal (ET) tube having a patient end that is sized to pass into a patient airway and a user end that is located opposite the patient end, the stylet comprising:
a tube portion having a tip located at a first end of the stylet and an inlet formed at the tip, wherein the tube portion is configured to be inserted into the ET tube and is sufficiently rigid to retain a selected shape after being inserted into the ET tube;
an elongate suction interface having a first longitudinal end connected to the tube portion and a second longitudinal end configured to interface with the suction system;
an outlet provided at the second longitudinal end of the suction interface;
a valve opening formed in the suction interface and positioned longitudinally between the first and second longitudinal ends of the suction interface;
a thumb valve having a tube surrounding the valve opening and extending away from the suction interface;
a thumb opening located at an end of the tube that is opposite the valve opening;
a first midline beginning at an intersection point within the suction interface and extending along a center of the suction interface and through a center of the outlet at the second longitudinal of the suction interface;
a second midline beginning at the intersection point and extending through a center of the valve opening and along a of the tube towards the thumb opening, wherein the intersection point is located at an intersection of the first midline with the second midline, and wherein an angle Θ taken at the intersection point and formed between the first and second midlines is less than 90 degrees
wherein the suction interface is arranged to direct a first ingoing airflow through the first longitudinal end and to the second longitudinal end in a first travel direction, to direct a second ingoing airflow through the tube and to the second longitudinal end in a second and different travel direction, and to direct an outgoing airflow out of the suction interface via the second longitudinal end and outlet in the first travel direction.

28. A stylet configured for insertion into and for use in guiding an endotracheal (ET) tube and for use in conjunction with a suction system to provide suction within the ET tube, the stylet comprising:
a tube portion having a tip located at a first end of the stylet and an inlet formed at the tip, wherein the tube portion is configured to be inserted into the ET tube and is sufficiently rigid to retain a selected shape after being inserted into the ET tube;
a suction interface consisting: of a first air inlet portion, a second air inlet portion, and a single air outlet end configured to interface with the suction system,
wherein the first air inlet is formed at a first longitudinal end of the suction interface, the air outlet is formed at a second longitudinal end of the suction interface and is collinear with the first air inlet portion, and the second air inlet is formed longitudinally between the first and second longitudinal ends of the suction interface,
wherein a first midline extends through the air outlet, a second midline extends through the second air inlet, and an angle Θ taken at an intersection point of the first and second midlines is less than 90 degrees.
